(19) United States
(12) Patent Application Publication
(10) Pub. No.: US 2015/0157701 A1
(43) Pub. Date: Jun. 11, 2015

(54) METHOD FOR ATTENUATING A BACTERIUM OF THE MYCOBACTERIUM TUBERCULOSIS COMPLEX FOR PRODUCING A TUBERCULOSIS VACCINE

(71) Applicant: FOUNDATION MEDITERRANEE INFECTION, Marseille Cedex 05 (FR)

(72) Inventors: Michel Drancourt, Marseille (FR); Otmane Lamrabet, Marseille (FR); Didier Raoult, Marseille (FR)

(73) Assignee: Fondation Mediterranee Infection, Marseille Cedex (FR)

(21) Appl. No.: 14/395,727

(22) PCT Filed: May 16, 2013
(86) PCT No.: PCT/FR2013/051078

§ 371 Date: Oct. 20, 2014

(30) Foreign Application Priority Data
May 21, 2012 (FR) 1254623

(51) Int. Cl. A61K 39/04; C07K 14/35; C12N 1/36

(52) U.S. Cl. CPC A61K 39/04; C12N 1/36; C07K 14/35; A61K 2039/522

(57) ABSTRACT

The present invention concerns the use of a strain of a mycobacterium of the Mycobacterium tuberculosis complex in which a mspA gene capable of expressing a porin A of Mycobacterium smegmatis has been inserted, to produce a vaccine for the prevention of infection with a bacterium of the Mycobacterium tuberculosis complex in a host having eukaryote cells, preferably macrophages, the said strain of mycobacteria thus transformed having reduced growth in the said eukaryote cells.

Tuberculosis is an infectious disease affecting man and animal due to one of the species of the Mycobacterium tuberculosis complex, namely and in particular Mycobacterium tuberculosis, Mycobacterium bovis (and the BCG strains derived therefrom), Mycobacterium africanum, Mycobacterium canettii, Mycobacterium caprae, Mycobacterium microti, and Mycobacterium pinnipedii [19] and Mycobacterium mungii (20). Tuberculosis causes about 9 million new cases of infection every year over the world and kills about 3 million people every year. The HIV infection epidemic coincided with the re-emergence of tuberculosis in developed countries and is accompanied by a worrying emergence of Mycobacterium tuberculosis strains resistant to first-line anti-tuberculosis antibiotics. Tuberculosis is a contagious disease and can be contracted from persons suffering from bacilliform pulmonary tuberculosis i.e. persons expelling living mycobacteria of the Mycobacterium tuberculosis complex in their expectoration.

For a long time, prevention against pulmonary tuberculosis was based on vaccination with Bacillus Calmette-Guérin (BCG) which is an attenuated strain of Mycobacterium bovis, a mycobacterium very closely related to Mycobacterium tuberculosis and also the cause of tuberculosis in man and animals, chiefly bovine tuberculosis. However the efficacy of the BCG vaccine varies considerably depending on clinical circumstances and the countries in which this efficacy has been measured. This efficacy varies between 0% protection and up to 60% protection. This poor protection by the BCG vaccine against pulmonary tuberculosis has led health authorities in several countries to abandoning this vaccination. This has been the case in France since July 2007 since BCG vaccination is no longer compulsory in France with the exception of some more particular groups of the population such as health care providers.

Several experimental studies have endeavoured to increase the protective nature of the BCG vaccine using different genetic engineering methods of the Mycobacterium tuberculosis genome, but up until now none of these constructs has allow the development of an alternative vaccine to BCG for protection against tuberculosis and in particular against pulmonary tuberculosis. One first strategy was to develop variants of BCG which produce and secrete cytokines [1]. A second strategy set out to construct recombinant BCG expressing an antigen protein of 30 kilo-daltons of Mycobacterium tuberculosis; this construct exhibited a protective effect in animal models. Finally several research studies engineered the genome of the BCG strain so that it secretes the listeriolysin of the bacterium Listeria monocytogenes to stimulate CD 8 and CD 4 lymphocytes involved in the protection against tuberculosis [2]. A last strategy turned to producing attenuated strains of Mycobacterium tuberculosis via inactivation of virulent genes such as the phoP gene and the mce gene [3, 4]. These constructs showed greater protection than BCG against infection with Mycobacterium tuberculosis in animal models [3, 4].

As is known per se such attenuated strains of Mycobacterium responsible for infection in man or animal are able to be used to produce vaccines, in particular by inserting said attenuated bacteria in the said vaccine.

As is known the strains of mycobacteria used as vaccines, and in particular the H37Rv strain, when cultured particularly in vivo in eukaryote cells have a survival time of no more than 30 days. In practice, a reduction in growth is accompanied by a decrease in the lifetime of the said bacterium in the said cells, particularly in vivo.

The MspA porin was discovered in membrane extracts of M. smegmatis as an oligomeric protein composed of 20 kDa sub-units [5]. It was considered to be the major and most abundant porin in M. smegmatis [5, 6]. Suppression of the mspA gene coding for MspA reduces the permeability of the external membrane of M. smegmatis to glucose [7], to phosphate [8], to metal ions and to amino acids [9] indicating that this porin is the main general diffusion pathway for different substrates in M. smegmatis, also allowing an inflow of nutrients from the medium outside the mycobacterium [8, 9]. The gene encoding the MspA porin is also detected in other fast-growth mycobacteria such as Mycobacterium chelonae [10] and Mycobacterium phlei [11]. It has been shown that the expression of the MspA porin in M. tuberculosis and M. bovis increases their susceptibility to antibiotics [12]. Similarly, the MspA porin of M. smegmatis increases the growth of a strain of M. bovis BCG in an axenic medium [14]. In these prior studies [12, 14], the authors determined minimum inhibitory concentrations of antibiotics for the control strain M. tuberculosis H37Rv and the strain expressing MspA by growth in an axenic Middlebrook 7H10 agar medium.

Finally, it has been shown that deletion of the mspA gene in M. smegmatis increases the survival of M. smegmatis in mouse macrophages (13).

It therefore follows from the literature that:

1—Different biological engineering techniques have been used to create mutations or deletions to construct attenuated strains of bacteria of the M. tuberculosis complex. Strategies to produce attenuated strains of Mycobacterium tuberculosis via inactivation pf virulent genes such as the phoP gene and mce gene [3, 4], have shown greater protection against infection with Mycobacterium tuberculosis than BCG in mouse and guinea pig animal models [3, 4]. However, these biological engineering techniques have not been validly used for the addition of genes absent from the genome of bacteria of the Mycobacterium tuberculosis complex for the purpose of use as vaccine or to produce a vaccine.

2—No prior art study has suggested that the expression of the mspA gene could attenuate the growth of a bacterium of the Mycobacterium tuberculosis complex in eukaryote cell models and animal models, and for still stronger reason that it could be used as vaccine or to produce a vaccine.

It is the objective of the present invention to provide novel strains of the M. tuberculosis complex having reduced growth rates whilst maintaining their immunogenicity.

Person skilled in the art of vaccines constantly seek to diversify vaccine production modes to improve the efficacy and reduce the side effects of the said vaccines, and also to extend the spectrum of mammals who could benefit from vaccination, in particular livestock such as cattle.

Unlike strategies which entail inactivating or removing genes to inactivate the mycobacterium so that it can be used for vaccine purposes, according to the present invention the inventors have surprisingly discovered that it is possible to attenuate a bacterium of the Mycobacterium tuberculosis complex through the addition and expression of a gene using molecular engineering methods.

To do so, the present invention provides a method for reducing the growth of a mycobacterium strain of the Mycobacterium tuberculosis complex in eukaryote cells, through the addition and expression in the said mycobacterium of a mspA gene coding for a porin A of Mycobacterium smegmatis with a view to use thereof as vaccine.

In Georgiana E. Purdy et al., 2009 (Molecular Microbiology. Vol 73. N^{o} 5. 1 Sep. 2009, pages 844-857) the authors used a CDC 1551 strain of Mycobacterium tuberculosis that is more virulent [31] than most other strains of M. tuberculosis and hence unsuitable for use as vaccine, and reported reduced viability of said transformed strain expressing the mspA porin in murine macrophages since the said murine macrophages were infected with a virus of murine leukaemia (MuLV), namely RAW 264.7 cells [28]. It is effectively known that, due to the phenomenon of viral interference, the infection of cells of the immunity system such as macrophages with a first virus (MuLV) leads to a reduction in the viability of a second microbe Mycobacterium tuberculosis infecting the said cells [29]. This is why the authors do not mention the advantage of the possible use of a said mutant of Mycobacterium tuberculosis to impart increased immunity to a host with a view to preparing a vaccine against tuberculosis.

In Mailaender et al. [12], the results of an increase in the growth of a transformed strain expressing the mspA porin compared with a wild-type strain of Mycobacterium tuberculosis in a culture medium, suggest to those skilled in the art that the strain thus transformed does not allow a decrease in the growth of the said strain in host eukaryote cells. In Mailaender et al. 2004 [12], the authors interpreted the experimental data they obtained solely along the line of a greater susceptibility of the Mycobacterium tuberculosis strain to natural antibiotics (peptides derived from ubiquitin) for the purpose of providing novel molecules having activity against tuberculosis and not with a view to providing a new vaccine.

According to the present invention, it has been discovered first by using macrophage lines not infected with a virus, and more especially by performing assays on cells of healthy human volunteer donors, that it is possible to obtain an improved attenuated strain of Mycobacterium tuberculosis which can advantageously be used as vaccine. These results were confirmed by in vivo experimental data in animal as reported in Example 4 below. These results, showing the innocuousness of the mutant strain according to the present invention, surpass all results currently published for BCG [30] in this field and form the basis for an improved vaccine against tuberculosis.

More specifically, the present invention provides the use of a mycobacterium strain of the Mycobacterium tuberculosis complex in which a mspA gene capable of expressing a porin A of Mycobacterium smegmatis has been inserted, for the production of a vaccine to prevent infection with a bacterium of the Mycobacterium tuberculosis complex in a host comprising eukaryote cells, preferably macrophages, the said strain of mycobacteria thus transformed showing reduced growth in the said eukaryote cells.

More particularly the subject of the present invention is the use of a transformed strain of Mycobacterium tuberculosis according to the invention to produce a vaccine for the prevention of tuberculosis in mammals or birds, the said cells being macrophages.

Further particularly, the subject of the present invention is the use of a strain transformed according to the invention that can be used to produce a vaccine for the prevention of human tuberculosis.

According to the present invention, it has been observed that:

a—A mycobacterium strain thus transformed of the Mycobacterium tuberculosis complex allowing the expression of the MspA porin in said mycobacterium of the M. tuberculosis complex exhibits stronger growth than the wild-type strain of Mycobacterium tuberculosis in a culture medium not comprising eukaryote cells (axenic medium)—in this respect the inventors have confirmed the results previously published in the literature [12, 14];

b—however, surprisingly, the growth of this transformed strain in co-culture with eukaryote cells allows a decrease in the growth of the Mycobacterium tuberculosis mutant compared with growth of the original wild-type strain, as demonstrated when using three different eukaryote cell culture media comprising in particular mouse macrophages and human macrophages. These macrophages are particularly involved in tuberculosis and in reactions of immunogenicity and hence of protection with regard to use as vaccine. Macrophages are human and animal cells which phagocyte Mycobacterium tuberculosis mycobacteria in an attempt to destroy them and to present the antigens of the mycobacteria to other cells of the immunity system [25].

This result of intracellular growth in co-cultures of eukaryote cells was fully unexpected since it opposes the finding obtained by the inventors and published in the international literature concerning cultures in axenic media not containing eukaryote cells [12, 14].

In addition, in vivo assays in mice allowed the demonstration that this reduction in growth is maintained in vivo and that the immunogenicity properties of the attenuated strain are also maintained, this therefore able to be given advantageous use for the production of a vaccine and even as vaccine for tuberculosis in mammals and birds.

More particularly, the said mspA gene is inserted in the said strain and placed under the control of a promoter allowing the expression of the said mspA gene in a said mycobacterium of the Mycobacterium tuberculosis complex.

Further particularly, a plasmid containing the said mspA gene is inserted under the control of a promoter for the expression of the said mspA gene in a said bacterium of the Mycobacterium tuberculosis complex.

Still further particularly, the said mspA gene and the said elements controlling the expression of the said mspA gene in a said bacterium of the Mycobacterium tuberculosis complex are inserted in the chromosome of the said bacterium of the Mycobacterium tuberculosis complex.

Herein by “bacteria of the Mycobacterium tuberculosis complex” is meant one of the bacteria of the species selected at least from among Mycobacterium tuberculosis, Mycobacterium bovis and the clones thereof or BCG sub-species, Mycobacterium africanum, Mycobacterium canettii, Mycobacterium caprae, Mycobacterium microti, Mycobacterium pinnipedii and Mycobacterium mongii. These species are all closely related, their genomes having more than 99% similarity for those species currently sequenced; they all cause the same clinical disease and cause the same tissue lesions in infected organs, characterized by the formation of granulomas with giant cells of macrophage type [26].

In one preferred embodiment of the invention, the said strain of the Mycobacterium tuberculosis complex is a strain of the species Mycobacterium tuberculosis.

More particularly, the said original strain of Mycobacterium tuberculosis is the H37Rv strain.

Further particularly, the said mspA gene comprises the sequence SEQ ID N^{o} 1 in the sequence listing appended to this description.

Still further particularly the said mspA gene is under the control of expression elements called <<hsp60 promoters>> consisting of SEQ. ID. N^{o} 4 in the sequence listing appended to the present description. Advantageously, the said eukaryote cells are mammalian or bird cells, preferably the said cells are human cells. More preferably the said cells are macrophage cells.

The present invention also provides a vaccine comprising a transformed strain of a bacterium of the Mycobacterium tuberculosis complex in which a said mspA gene has been inserted capable of expressing porin A of Mycobacterium smegmatis in a said mycobacterium allowing the implementation of the method of the invention.

More particularly it is a transformed strain of Mycobacterium tuberculosis.

Further particularly, it is the strain called H37RvpVVMspA hereinafter, deposited on 26 Apr. 2012 with the NCTC collection (National Collection of Type Cultures), Health Protection Agency Culture Collections, Porton Down, Salisbury Wiltshire, SP4 0JG UK, under number 12042601. This deposit was submitted in accordance with the Treaty of Budapest.

A <<transformed strain>> according to the invention more particularly exhibits reduced growth of the said bacterium in eukaryote cells by a least 90%, even at least 99% in terms of number of bacteria per ml of cell culture. In other words, the number of bacteria per ml of cell culture is divided by at least 10, even divided by at least 100.

A mycobacterium strain transformed according to the invention has a survival term in eukaryote cell culture and in particular in vivo of no more than 60 days and preferably no more than 50 days.

In practice, the reduction in growth is accompanied by a decrease in the lifetime of the said bacterium in the said cells, in vivo in particular, of at least 3 to 10 days

Other characteristics and advantages of the present invention will become better apparent on reading the following examples given with reference to FIGS. 1 to 3 in which:

FIG. 1 is a SDS-PAGE gel photograph showing the level of expression of the MspA porin. Lane M shows the molecular weight markers. Lane 1 shows the separation of an extract of proteins of M. smegmatis, lane 2 an extract of proteins of M. tuberculosis H37Rv/pVV16 and lane 3 shows the separation of an extract of proteins of M. tuberculosis H37Rv/pVVMspA, the MspA protein being seen at 20 KDa.

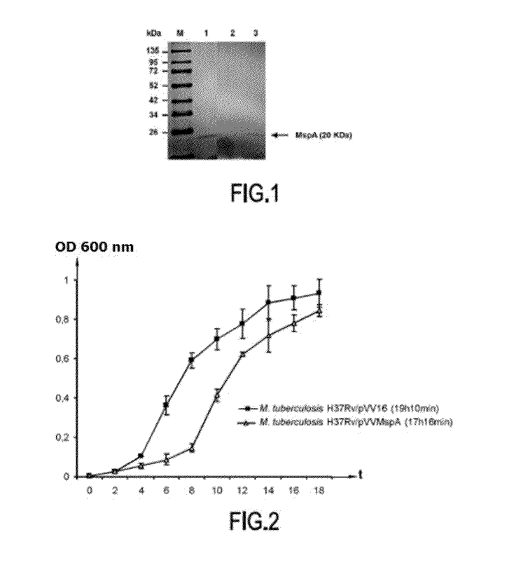

FIG. 2 gives the curves of the increase in growth of M. tuberculosis in the presence of the MspA porin on the 7H9 axenic media of Example 1, measured along the Y-axis in optical density (OD) at 600 nm, from 0 to 1, for 18 days (“t” on the X-axis).

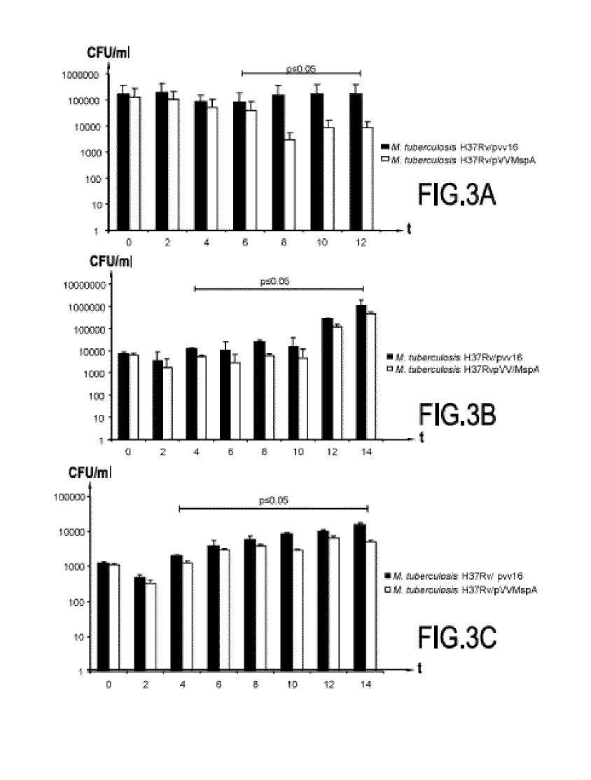

FIGS. 3A to 3C give intracellular growth graphs of M. tuberculosis H37Rv/pVV16 and M. tuberculosis H37Rv/MspA in different eukaryote cells: A. polyphaga (FIG. 3A), BMDMs (FIG. 3B) and hMdMs (FIG. 3C) in Examples 2 and 3, measured in number of CFU/ml of cell lysate, from 0 to 1000000, (Y-axis) after 0 to 14 days of culture (“t” on the X-axis).

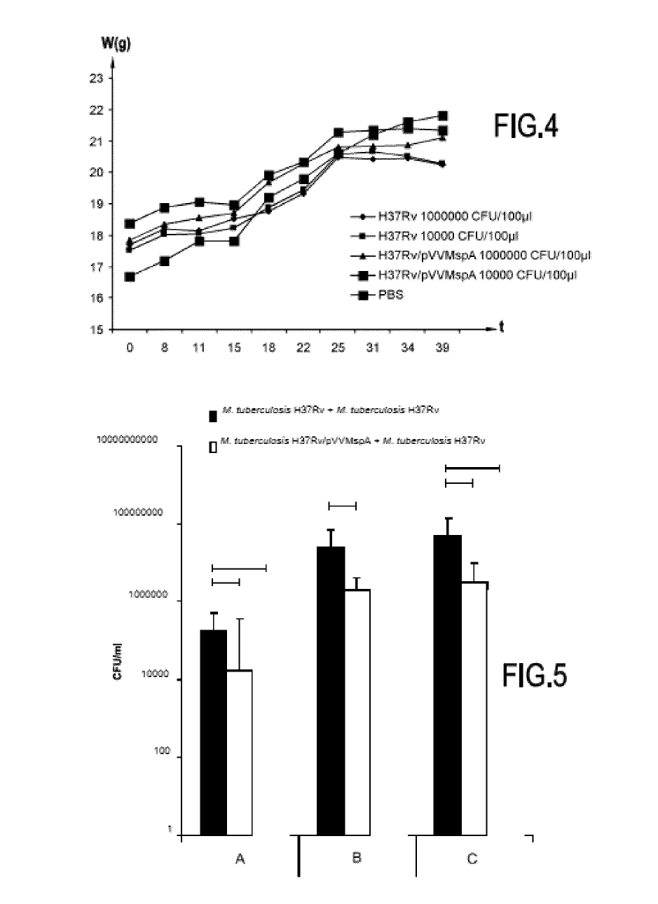

FIG. 4 shows the trend in weight (w) of mice in grams (g) as a function of time (t), in number of days, from 0 to 39, for mice infected with the wild-type H37Rv strain and for mice infected with the modified H37Rv/pVVMspA strain, at different concentrations (10 000 CFU/100 μl and 10 000 CFU/100 μl).

FIG. 5 shows the bacteriaL load (number of CFUS) in the liver (A), spleen (B) and lungs (C), after inoculation with the wild-type strain of M. tuberculosis H37Rv (▪) and with the M. tuberculosis strain according to the present invention (□), such as discussed in Example 4.

### EXAMPLE 1

### Growth of Mycobacterium Tuberculosis Expressing MspA in an Axenic Medium

Initially, the inventors amplified the gene coding for the MspA porin from the genome of Mycobacterium smegmatis, and then inserted the amplification product in the expression vector pVV16 [16] which is characterized by the presence of two genes coding for proteins resistant to hygromycin and kanamycin (selection markers) to obtain the plasmid pVVMspA. The inventors subsequently incorporated the pVVMspA plasmid via electroporation into strain HS7Rv of Mycobacterium tuberculosis and selection of the transformed clones was conducted using hygromycin and kanamycin as antibiotics.

Experimental Protocols

1) Construction of the pVVMspA Plasmid:

First the inventors extracted genomic DNA from the M. smegmatis strain mc155 (ATCC 700084) and used standard PCR to amplify the gene encoding the MspA porin [5].

The sequence gi_{—}118467340 of the said gene is the following sequence SEQ. ID. N^{o} 1:

5′-ATGAAGGCAATCAGTCGGGTGCTGATCGCGATGGTTGCAGCCATCG
CGGCGCTTTTCACGAGCACAGGCACCTCTCACGCAGGCCTGGACAACGA
GCTGAGCCTCGTTGATGGCCAGGACCGCACCCTCACCGTGCAGCAGTGG
GACACCTTCCTCAATGGTGTGTTCCCCCTGGACCGCAACCGTCTTACCC
GTGAGTGGTTCCACTCCGGTCGCGCCAAGTACATCGTGGCCGGCCCCGG
TGCCGACGAGTTCGAGGGCACGCTGGAACTCGGCTACCAGATCGGCTTC
CCGTGGTCGCTGGGTGTGGGCATCAACTTCAGCTACACCACCCCGAACA
TCCTGATCGACGACGGTGACATCACCGCTCCGCCGTTCGGCCTGAACTC
GGTCATCACCCCGAACCTGTTCCCCGGTGTGTCGATCTCGGCAGATCTG
GGCAACGGCCCCGGCATCCAGGAAGTCGCAACGTTCTCGGTCGACGTCT
CCGGCGCCGAGGGTGGCGTGGCCGTGTCGAACGCCCACGGCACCGTGAC
CGGTGCGGCCGGCGGTGTGCTGCTGCGTCCGTTCGCCCGCCTGATCGCC
TCGACCGGTGACTCGGTCACCACCTACGGCGAACCCTGGAACATGAACT
GA-3′.

The primers used were:

MSPA-pVV16F:
(SEQ. ID. N^{o} 2)
5′-cccccccatatgaaggcaatcagtc-3′,
and
MSPA-pVV16ndeI:
(SEQ. ID. N^{o} 3)
5′-ccccatatgtcagttcatgttccaggg-3′.

These primers were determined to generate a PCR product corresponding to the entirety of the mspA gene (636 bp) hosting a cleavage site on each side for the restriction enzyme Ndel (this enzymatic cleavage site is underlined in the above primer sequences) to allow cloning in the expression vector pVV16; followed by constitutive expression thereof.

Like other expression vectors such as pMS3 [15], pMV261 [16], the pVV16 vector has a hygromycin resistance cassette and an hsp60 expression promoter (SEQ. ID. N^{o} 4) in the mycobacteria [16]. The specificity of pVV16 is an expression promoter in Escherichia coli (used for verification of the plasmid construct at the time of cloning) and a kanamycin resistance cassette allowing dual section [16].

Next the PCR fragment was cloned at the Ndel site of the pVV16 plasmid to give the pVVMspA plasmid. This latter was inserted in Escherichia coli DH5 alpha using the most simple means: heat shock. The plasmid construct was verified by enzymatic digestion and PCR amplification followed by sequencing.

The SEQ. ID. N^{o} 4 sequence of the hsp60 promoter is
5′-AACGCGTCGGCGGCTGCGCGCACCGAGTCCAGCGAGCACAGATCGAGT
TGCTGCAGCGTGACGTGGGCGCCTGGGCGGGCGGCCATGATGCGGGCCCGG
GCGGCGTTGCCCTTCTCGAGATTGCGGACGGCCAAACCTACGTGTGCACCG
CGGTCGGCAAACACGGCGGCGGTGTGGTAGCCGATGCCGGTGTTGGCGCCG
GTGACCACAACGACGCGCCCGCTTTGATCGGGGACGTCTGCGGCCGACCAT
TTACGGGTCTTGTTGTCGTTGGCGGTCATGGGCCGAACATACTCACCCGGA
TCGGAGGGCCGAGGACAAGGTCGAACGAGGGGCATGACCCGGTGCGGGGCT
TCTTGCACTCGGCATAGGCGAGTGCTAAGAATAACGTTGGCACTCGCGACC
GGTGAGTCGTAGGTCGGGACGGTGAGGCCAGGCCCGTCGTCGCAGCGAGTG
GCAGCGAGGACAACTTGAGCCGTCCGTCGCGGGCACTGCGCCCGGCCAGCG
TAAGTAGCGGGGTTGCCGTCACCCGGTGACCCCCGGTTTCATCCCCGATCC
GGAGGAATCACTTCGCA-3′

The expression cassette of the mspA gene under the control of the hsp60 promoter consists of the juxtaposition of SEQ. ID. N^{o} 4 and SEQ. ID. N^{o} 1.

2) Preparation of Electrocompetent Mycobacterium Tuberculosis Mycobacteria and Insertion of the pVVMspA Plasmid:

The electrocompetent (permeable to the insertion of a plasmid) Mycobacterium tuberculosis bacteria strain H37Rv (ATCC 27294) [17] were prepared as previously described by Van Kessel and Hatfull [18]. In brief, the Mycobacterium tuberculosis mycobacteria were harvested from a liquid culture of M. tuberculosis H37Rv of optical density (OD₆₀₀) between 0.8-1.0, washed three times in 10% glycerol and re-suspended in the same buffer. The plasmids pVV16 (control to ensure that changes in growth were not due to the expression vector itself but indeed to the presence of the mspA gene) and pVVMspA (plasmid containing the gene of the MspA porin of interest) were inserted separately via electroporation into the mycobacteria using an electroporator (reference Multiporator/Electroporator 2510, Le Pecq, France) at 1000 omega, 2.5 kV and 25 uF. The electroporated mycobacteria were collected in 1 mL of enriched liquid Middelbrook 7H9 medium (Sigma-Aldrich, Lyon, France), placed in culture at 37° C. for 12 h then spread over Middelbrook 7H10 medium made selective through the addition of kanamycin plus hygromycin, and incubated at 37° C. for 3 to 4 weeks. The transcription and translation of the mspA gene in the transformed M. tuberculosis mycobacteria were verified by PCR, RT-PCR and SDS-PAGE.

The MspA porin is characterized by its stability against denaturing by heat [5]. In FIG. 1, SDS-PAGE gel electrophoresis was performed using protein extracts of M. tuberculosis H37Rv/pVV16 (lane 2), M. tuberculosis H37Rv/pVVMspA (lane 3) and M. smegmatis (positive control) (lane 1). These extracts were prepared by high temperature selective extraction [27]. FIG. 1 shows the expression of the mspA gene in M. tuberculosis H37Rv and in the control strain M. smegmatis. The detected protein spots were excised from the gel and analysed by MALDI-TOF mass spectrometry. The protein spots were identified with sequence coverage ranging from 21% to 29% and similarity with the MspA protein sequence of M. smegmatis of 20 KDa.

3) Growth in liquid medium of the transformed or non-transformed M. tuberculosis strains: M. tuberculosis H37Rv/pVV16 (control transformed strains not expressing the MspA porin) and M. tuberculosis H37Rv/pVVMspA (transformed strains expressing the MspA porin) were placed in pre-cultures in 5 mL of Middlebrook 7H9 medium (Becton Dickinson, Le Pont de Claix, France) for 15 days until a DO₆₀₀ value of 1 was obtained. The mycobacteria were then vigorously vortex mixed under agitation to remove cell clusters, harvested and diluted in 10 ml of fresh 7H9 medium. The growth rates were determined for both strains in three independent cultures by DO₆₀₀ measurement using a BACTEC MGIT 960 system (Becton Dickinson, Le Pont de Claix, France). This fully automated system is based on the detection of oxygen consumption by aerobic bacteria.

4) Results.

To study the influence of the MspA protein on the growth of M. tuberculosis, the inventors performed in vitro growth experiments with the strains M. tuberculosis/pVV16 and M. tuberculosis/pVVMspA in liquid 7H9 axenic medium in three independent cultures. The inventors used a suspension of cells and growth of the bacteria was measured in the BACTEC MGIT 960 system for 17 days. The generating times of the control strain M. tuberculosis/pVV1 and of the M. tuberculosis/pVVMspA strain were respectively 19 h 10 min±10 min and 17 h 16 min±12 min (FIG. 2). It was therefore ascertained by the inventors that the strain which expresses the MspA porin shows much faster growth than the control strain (p 0.05). This experiment, performed three times, showed that the expression of the MspA porin increases the growth rate of M. tuberculosis.

### EXAMPLE 2

### Growth of Mycobacterium Tuberculosis Expressing MspA in Co-Culture with the Amoeba A. Polyphaga

1) Experimental Protocol:

Ten millilitres of a suspension of amoeba (˜10⁵ amoeba/ml) were inoculated with ˜10⁶ mycobacteria/ml (MOI=10). As controls, A. polyphaga and the mycobacteria were cultured separately in PAS buffer (Page's modified Neff's amoeba saline) (21, 22, 23) After an incubation time of 24 h at 32° C., the co-culture was washed three times with PAS and incubated at 32° C. in 10 ml PAS for 10 to 15 minutes.

2) Results:

Using amoeba as eukaryote cells, it was observed that the two strains M. tuberculosis H37Rv/pVV16 and M. tuberculosis H37Rv/pVVMspA survived the experiment, but the number of M. tuberculosis H37Rv/pVVMspA mycobacteria which expressed the MspA porin was significantly reduced (p 0.05; Student's statistical test) after 8 days of co-culture. At the 12^{th} day, the number of colonies was 2×10⁵±6×10⁴ for M. tuberculosis H37Rv/pVV16 against 8×10^{3±5×10}³ for M. tuberculosis H37Rv/pVVMspA (FIG. 3A).

FIG. 3A gives the intracellular growth graphs of M. tuberculosis H37Rv/pVV16 and M. tuberculosis H37Rv/MspA in a culture of eukaryote cells: A. polyphaga.

These data indicate a reduction of about two logs for the intracellular growth of M. tuberculosis H37Rv/pVVMspA compared with the M. tuberculosis H37Rv/pVV16 strain, i.e. a reduction of about 99% in the number of bacteria/ml of culture.

### EXAMPLE 3

### Growth of Mycobacterium Tuberculosis Expressing MspA in Co-Culture with Human Macrophages (hMdMs) or Mouse Macrophages (BMDMs)

1) Experimental Protocol:

The BMDMs [24] and hMdMs isolated from leukopacks (Etablissement Français du Sang, Marseille, France) using a Ficoll gradient (MSL, Eurobio, Courtaboeuf, France) and then differentiated were seeded separately (10⁵ cells/well) in 24-well culture plates in RPMI 1640 medium containing 10% FCS. The BMDMs and hMdMs were separately infected with M. tuberculosis, M. tuberculosis H37Rv/pVV16 and M. tuberculosis H37Rv/pVVMspA for 24 hours and the macrophages were then washed to remove free mycobacteria before being incubated for different periods of time in RPMI 1640 medium containing 10% foetal calf serum (FCS). As controls, each cell type and the mycobacteria were cultured separately.

Determination of the Number of Intracellular Mycobacteria (CFUs):

At a given time the infected cells were lysed with 0.1% sodium dodecyl sulfate (SDS) for 30 min then passed through a 26-gauge needle to ensure complete lysis. The lysate (500 μL) was washed three times with PBS, spread over solid 7H10 medium (Becton Dickinson, Le Pont de Claix, France) and incubated for 15 days at 37° C. to determine the number of colonies of intracellular mycobacteria (CFUs). All the experiments were conducted in triplicate.

2) Results:

By using BMDM macrophages as eukaryote cells, the inventors observed that the two strains M. tuberculosis H37Rv/pVV16 and M. tuberculosis H37Rv/pVVMspA survived throughout the experiment time (14 days). It was observed by the inventors that between the 4^{th} and 14^{th} day the control strain H37Rv/pVV16 showed significantly greater growth than the strain expressing MspA (p≦0.05) (FIG. 3B). At the 14^{th} day the number of colonies was 1×10⁶±7×10⁵ for M. tuberculosis H37Rv/pVV16 against 4×10⁵±1 10⁵ for M. tuberculosis H37Rv/pVVMspA (p 0.05).

Co-Culture of Transformed or Non-Transformed M. Tuberculosis with Human Macrophages.

Using hMdM macrophages as eukaryote cells, the inventors observed that on and after the 4^{th} day and up until the end of the experiment, the control strain M. tuberculosis H37Rv/pVV16 showed significantly greater growth than the strain expressing MspA (p 0.05) (FIG. 3C). At Day 14 the number of colonies was 2×10⁴±2×10³ for M. tuberculosis H37Rv/pVV16 against 5×10³±7 10² for M. tuberculosis H37Rv/pVVMspA.

To conclude, these results indicate that the expression of the MspA porin in M. tuberculosis leads to difficult multiplication of the bacterium in the 3 types of tested eukaryote cells compared with the control strain.

FIGS. 3B and 3C give the intracellular growth graphs of M. tuberculosis H37Rv/pVV16 and M. tuberculosis H37Rv/MspA in different cultures of macrophages: BMDMs (FIG. 3B) and hMdMs (FIG. 3C).

These data indicate a decrease of about one log in the intracellular growth of M. tuberculosis H37Rv/pVVMspA compared with the M. tuberculosis H37Rv/pVV16 strain, i.e. a decrease of about 90% in the number of bacteria/ml of culture.

### EXAMPLE 4

### Growth of Mycobacterium Tuberculosis Expressing MspA in an Animal Model and Protection Against Tuberculosis

1) Experimental Protocol:

The study in animals was performed in an NSB laboratory 3 days after receiving approval from the local office of the ethics committee for animal experimentation. 6- to 8-week old Balb/c mice (10 per group) were infected via intraperitoneal route with 100 μL of bacterial culture. Tw different inocula of each of the above strains (wild-type H37Rv ATCC 27294 strain and mutant M. tuberculosis H37Rv/pVVMspA strain) were used: 1×10⁷ CFU/mL and 1×10⁵ CFU/mL. As control, a group of mice was infected with 100 μL of sterile PBS. The weight, performance and survival of the mice were monitored for 1 month.

Examination of the infected mice was carried out in accordance with a protocol published by other researchers [3]. In addition, at a first phase some surviving mice were sacrificed, the spleen, liver and lungs were analysed by pathological anatomy and the bacterial load was determined per gram of tissue, per culture and expressed in CFUs. At a second phase the surviving mice in the two groups were re-inoculated with 10⁷ CFU/mL of M. tuberculosis h37Rv and the same observation protocol was followed.

2) Results:

Current data show that the mice infected with the modified strain of the invention have fewer behavioural problems and have a faster weight gain than the mice infected with the wild-type strain of M. tuberculosis (FIGS. 4 and 5).

Examination of the infected mice was conducted following a protocol published by other researchers [3]. Ten weeks after infection, all the mice inoculated with the mutant M. tuberculosis H37Rv/pVVMspA strain had survived, whereas those inoculated with the wild-type strain showed a mortality rate of 50%. This survival was correlated with a very low number of colony forming units (CFUs) of M. tuberculosis H37Rv/pVVMspA in the lungs. The mice inoculated with the mutant M. tuberculosis H37Rv/pVVMspA strain developed few tissue lesions comprising small granuloma. One mouse inoculated with the wild-type M. tuberculosis H37Rv strain showed an abdominal fistula. Macroscopic analysis showed that the weight of the spleen in the mice inoculated with the wild-type M. tuberculosis H37Rv strain was significantly greater than that of mice inoculated with the mutant strain. In addition, the number of granulomas was respectively 24±21 vs. 0 in the liver; and 61±27 vs. 8±7 in the spleen, these differences being significant (p<0.05). In the spleen, the count of mycobacteria showed a significant reduction in the number of CFUs for the mutant M. tuberculosis H37Rv/pVVMspA strain compared with the wild-type M. tuberculosis H37Rv strain. Immunogenicity tested in cell assays was preserved.

90 days after the first inoculation, the inventors again inoculated the two groups of mice with the wild-type Mycobacterium tuberculosis H37RV strain using an inoculum of 10⁷ CFU per ml. The mice were again monitored for 90 days; all the mice survived with the exception of one mouse out of every 4 inoculated with the wild-type strain at the first inoculation.

In addition, the weight gain was significantly greater (P<0.05) in the group of mice initially inoculated with the vaccine strain according to the present invention compared with the mice initially inoculated with the wild-type strain, as observed on and after the 38th inoculation day (p=Student's statistical test which indicates the probability of error (here <5%) when asserting that 2 means of 2 series are different).

The mice were euthanized 90 days after the second inoculation; histo-pathological analysis of the tissues showed a significantly higher number of nodules of lung granulomas in the group initially inoculated with the wild-type tuberculosis (33±37 nodules) compared with the mice initially inoculated with the vaccine strain of the present invention (21±25 nodules) (p<0.05).

Finally the bacterial load (number of CFUs) of Mycobacterium tuberculosis H37RV was significantly higher in the group of mice initially inoculated with the wild-type tuberculosis than in the group of mice initially inoculated with the vaccine strain of the present invention (p<0.05) (FIG. 5).

The inventors therefore obtained three types of elements of proof: a clinical element of proof (mortality); a histological element of proof (number of granulomas per organ); and a microbiological element of proof (bacterial load after inoculation) allowing the conclusion to be drawn that the M. tuberculosis H37RV/PVVMSPA mutant strain imparts protection in this animal model against wild-type tuberculosis.

These results showing the innocuousness of the mutant strain of the present invention and the protective nature of the M. tuberculosis mutant strain of the present invention against infection with M. tuberculosis, surpass all the results currently published for BCG [30] in this field, to form the basis of an improved vaccine against tuberculosis.

In Brandt L. et al 2002 [30], no protection against mortality induced by tuberculosis is shown 6 weeks after infection with M. tuberculosis, although the prospect of its infection in man is of capital importance. In addition, the bacterial loads of M. tuberculosis are very moderately reduced in the spleen and lungs.

### BIBLIOGRAPHICAL REFERENCES

[1] Metchock B G, Nolte F S, Wallace R J Jr. Mycobacterium. In: Murray P R, Jo Baron H, Pfaller M A, Tenover F C, Yolken R H, Eds. Manual of clinical microbiology. ASM Press; Washington, D.C., 1999; 399-437.
[2] Grode L, Seiler P, Baumann S, Hess J, Brinkmann V, Nasser Eddine A, Mann P, Goosmann C, Bandermann S, Smith D, Bancroft G J, Reyrat J M, van Soolingen D, Raupach B, Kaufmann S H. Increased vaccine efficacy against tuberculosis of recombinant Mycobacterium bovis bacille Calmette-Guérin mutants that secrete listeriolysin. J Clin Invest. 2005; 115:2472-9.
[3] Gioffre A, Infante E, Aguilar D, Santangelo M P, Klepp L, Amadio A, Meikle V, Etchechoury I, Romano M I, Cataldi A et al. Mutation in mce operons attenuates Mycobacterium tuberculosis virulence. Microbes Infect 2005; 7:325-334;
[4] Martin C, Williams A, Hernandez-Pando R, Cardona P J, Gormley E, Bordat Y, Soto C Y, Clark S O, Hatch G J, Aguilar D et al. The live Mycobacterium tuberculosis phoP mutant strain is more attenuated than BCG and confers protective immunity against tuberculosis in mice and guinea pigs. Vaccine 2006; 24:3408-3419
[5] Niederweis, M., Ehrt, S., Heinz, C., Klocker, U., Karosi, S., Swiderek, K. M., Riley, L. W. and Benz, R. Cloning of the mspA gene encoding a porin from Mycobacterium smegmatis. Mol. Microbiol. 1999; 5:933-945.
[6] Ojha A, Hatfull G F. The role of iron in Mycobacterium smegmatis biofilm formation: the exochelin siderophore is essential in limiting iron conditions for biofilm formation but not for planktonic growth. Mol Microbiol. 2007; 66:468-83.
[7] Stahl, C. Kubetzko, S. Kaps, I. Seeber, S. Engelhardt, H. Niederweis, M. MspA provides the main hydrophilic pathway through the cell wall of Mycobacterium smegmatis. Mol Microbiol. 2001; 40; 451-64.
[8] Wolschendorf F, Mahfoud M, Niederweis M. Porins are required for uptake of phosphates by Mycobacterium smegmatis. J Bacteriol. 2007; 189:2435-42.
[9] Stephan J, Bender J, Wolschendorf F, Hoffmann C, Roth E, Mailäander C, Engelhardt H, Niederweis M. The growth rate of Mycobacterium smegmatis depends on sufficient porin-mediated influx of nutrients. Mol Microbiol. 2005; 58:714-30.
[10] Trias, J., Jarlier, V., and Benz, R. Porins in the cell wall of mycobacteria. Science 1992; 258: 1479-1481
[11] Riess, F. G., Dorner, U., Schiffler, B., and Benz, R. Study of the properties of a channel-forming protein of the cell wall of the Gram-positive bacterium Mycobacterium phlei. J Membr Biol 2001; 182: 147-157
[12] Mailaender C, Reiling N, Engelhardt H, Bossmann S, Ehlers S, Niederweis M. The MspA porin promotes growth and increases antibiotic susceptibility of both Mycobacterium bovis BCG and Mycobacterium tuberculosis. Microbiology 2004; 150:853-864
[13] Fabrino D L, Bleck C K, Anes E, Hasilik A, Melo R C, Niederweis M, Griffiths G, Gutierrez M G. Porins facilitate nitric oxide-mediated killing of mycobacteria. Microbes Infect. 2009; 11:868-75.
[14] Sharbati-Tehrani S, Meister B, Appel B, Lewin A. The porin MspA from Mycobacterium smegmatis improves growth of Mycobacterium bovis BCG. Int J Med Microbiol. 2004; 294:235-45.
[15] Scholz, O., Thiel, A., Hillen, W. & Niederweis, M. Quantitative analysis of gene expression with an improved green fluorescent protein. Eur J Biochem 2000; 267, 1565-1570.
[16] Stover C K, De la Cruz V F, Fuerst T R, Burlein J E, Benson L A, Bennett L T, Bansal G P, Young J F, Lee M H, Hatfull G F, Snapper S B, Barletta R G, Jacobs W R, Bloom B R. New use of BCG for recombinant vaccines. Nature 1991: 351:456-460.
[17] Cole S T, Brosch R, Parkhill J, Garnier T, Churcher C, Harris D, Gordon S V, Eiglmeier K, Gas S, Barry C E, 3rd et al: Deciphering the biology of Mycobacterium tuberculosis from the complete genome sequence. Nature 1998, 393(6685):537-544)
[18] Van Kessel, J. C., and Hatfull, G. F. (2007) Recombineering in Mycobacterium tuberculosis. Nat Methods 4: 147-152.
[19] Dye C. et al. Prospects for worldwide tuberculosis control under the WHO DOTS strategy. Lancet 1998; 352:1886-91.3.
[20] Alexander K A, Laver P N, Michel A L, Williams M, van Heiden P D, Warren R M, Gey van Pittius N C. Novel Mycobacterium tuberculosis complex pathogen, M. mungi. Emerg Infect Dis. 2010; 16:1296-9.
[21] Rowbotham, T. J. Isolation of Legionella pneumophila from clinical specimens via amoebae, and the interaction of those and other isolates with amoebae. J Clin Pathol 1983; 36: 978986.
[22] La Scola, B., Mezi, L., Weiller, P. J., and Raoult, D. Isolation of Legionella anisa using an amoebic coculture procedure. J Clin Microbiol 2001; 39:365-366.
[23] Greub, G., and Raoult, D. Microorganisms resistant to free-living amoebae. Clin Microbiol Rev 2004; 17: 413-433).
[24] Cook, P., Totemeyer, S., Stevenson, C., Fitzgerald, K. A., Yamamoto, M., Akira, S. et al. Salmonella-induced SipB-independent cell death requires Toll-like receptor-4 signaling via the adapter proteins. Tram and Trif. Immunology 2007; 122: 222-229).
[25] Gengenbacher M, Kaufmann S H. Mycobacterium tuberculosis: success through dormancy. FEMS Microbiol Rev. 2012; 36:514-32
[26] Isaza R. Tuberculosis in all taxa. In: Fowler M E, Miller R E, eds. Zoo and Wild Animal Medicine. 5^{th} ed. St. Louis, Mo.: Elsevier; 2003: 689-696
[27] Heinz, C., and Niederweis, M. Selective extraction and purification of a mycobacterial outer membrane protein. Anal Biochem 2000; 285;113-120
[28] HARTLEY J. W. et al. Retrovirology 2008, 5:1
[29] Microbiologie Médicale .Jawetz E. Melnick J L, Adelberg E A. Maloine S A Editor, 1973, p 384
[30] Brandt L. et al. Infect. Immun. 2002; 70: 672-678
[31] Manca C. et al, J. Immunol. 1999; 62: 6740-6

1. Method of producing a vaccine for the prevention of infection with a bacterium of the Mycobacterium tuberculosis complex in a host having eukaryote cells wherein a mycobacterium of the Mycobacterium tuberculosis complex is transformed in inserting in its chromosome a mspA gene capable of expressing a porin A of Mycobacterium smegmatis so that the thus transformed mycobacterium has reduced growth in said eukaryote cells.

2. The method according to claim 1 wherein a vaccine for the prevention of tuberculosis in mammals or birds is produced, said eukaryote cells being macrophages.

3. The method according to claim 2 wherein a vaccine for the prevention of tuberculosis in humans is produced.

4. The method according to claim 1, characterized in that said mspA gene is placed under the control of a promoter allowing the expression of the said mspA gene in said mycobacterium of the Mycobacterium tuberculosis complex.

5. The method according to claim 4, characterized in that said mspA gene and said promoter are inserted in a plasmid.

6. The method according to claim 1, characterized in that said mycobacterium of the Mycobacterium complex is Mycobacterium tuberculosis.

7. The method according to claim 6, characterized in that the origin strain of said Mycobacterium tuberculosis is the H37Rv strain.

8. The method according to claim 1, characterized in that said mspA gene comprises the sequence SEQ ID N^{o} 1.

9. The method according to claim 4, characterized in that said mspA gene is under the control of expression elements of the hsp60 promoter consisting of SEQ. ID. N^{o} 4.

10. The method according to claim 1, characterized in that a strain of said mycobacterium was deposited with the NCTC collection (UK) on 26 Apr. 2012, under number 12042601.

11. A vaccine obtained with the method according to claim 1, characterized in that it comprises said transformed mycobacterium of the Mycobacterium tuberculosis complex in which a mspA gene has been inserted capable of expressing a MspA porin of Mycobacterium smegmatis in the said mycobacterium.

12. The vaccine according to claim 11, characterized in that the said mycobacterium is Mycobacterium tuberculosis.

13. The vaccine according to claim 12, characterized in that a strain of said mycobacterium was deposited with the NCTC collection (UK) on 26 Apr. 2012, under number 12042601.


SEQUENCE LISTING (4 sequences)

SEQ ID NO: 1 - DNA, 636 bp, Artificial Sequence
  mspA gene of Mycobacterium smegmatis
atgaaggcaatcagtcgggtgctgatcgcgatggttgcagccatcgcggcgcttttcacgagcacaggcacctctcacgcaggcctggacaacgagctgagcctcgttgatggccaggaccgcaccctcaccgtgcagcagtgggacaccttcctcaatggtgtgttccccctggaccgcaaccgtcttacccgtgagtggttccactccggtcgcgccaagtacatcgtggccggccccggtgccgacgagttcgagggcacgctggaactcggctaccagatcggcttcccgtggtcgctgggtgtgggcatcaacttcagctacaccaccccgaacatcctgatcgacgacggtgacatcaccgctccgccgttcggcctgaactcggtcatcaccccgaacctgttccccggtgtgtcgatctcggcagatctgggcaacggccccggcatccaggaagtcgcaacgttctcggtcgacgtctccggcgccgagggtggcgtggccgtgtcgaacgcccacggcaccgtgaccggtgcggccggcggtgtgctgctgcgtccgttcgcccgcctgatcgcctcgaccggtgactcggtcaccacctacggcgaaccctggaacatgaactga

SEQ ID NO: 2 - DNA, 25 bp, Artificial Sequence
  Primer
cccccccatatgaaggcaatcagtc

SEQ ID NO: 3 - DNA, 27 bp, Artificial Sequence
  Primer
ccccatatgtcagttcatgttccaggg

SEQ ID NO: 4 - DNA, 575 bp, Artificial Sequence
  Sequence of hsp60 promoter
aacgcgtcggcggctgcgcgcaccgagtccagcgagcacagatcgagttgctgcagcgtgacgtgggcgcctgggcgggcggccatgatgcgggcccgggcggcgttgcccttctcgagattgcggacggccaaacctacgtgtgcaccgcggtcggcaaacacggcggcggtgtggtagccgatgccggtgttggcgccggtgaccacaacgacgcgcccgctttgatcggggacgtctgcggccgaccatttacgggtcttgttgtcgttggcggtcatgggccgaacatactcacccggatcggagggccgaggacaaggtcgaacgaggggcatgacccggtgcggggcttcttgcactcggcataggcgagtgctaagamtaacgttggcactcgcgaccggtgagtcgtaggtcgggacggtgaggccaggcccgtcgtcgcagcgagtggcagcgaggacaacttgagccgtccgtcgcgggcactgcgcccggccagcgtaagtagcggggttgccgtcacccggtgacccccggtttcatccccgatccggaggaatcacttcgca